(19) United States
(12) Patent Application Publication
(10) Pub. No.: US 2021/0322364 A1
(43) Pub. Date: Oct. 21, 2021

(54) TOPICAL FORMULATIONS AND METHODS OF USE THEREOF

(71) Applicant: MOMED Inc., Wayne, PA (US)

(72) Inventors: Ursula K. Bonner, Bryn Mawr, PA (US); Dennis J. Bonner, New Hope, PA (US)

(21) Appl. No.: 17/066,583

(22) Filed: Oct. 9, 2020

Related U.S. Application Data
(60) Provisional application No. 62/913,355, filed on Oct. 10, 2019.

(51) Int. Cl. A61K 31/352; A61K 36/185; A61K 31/167; A61K 31/165; A61K 9/00; A61K 31/164; A61K 36/28; A61K 31/10; A61K 36/53; A61K 9/107; A61K 31/05; A61K 47/46; A61K 47/12; A61K 47/10

(52) U.S. Cl. CPC A61K 31/352; A61K 36/185; A61K 31/167; A61K 31/165; A61K 9/0014; A61K 31/164; A61K 47/10; A61K 31/10; A61K 36/53; A61K 9/107; A61K 31/05; A61K 47/46; A61K 47/12; A61K 36/28

(57) ABSTRACT

Provided herein are topical pharmaceutical compositions comprising lidocaine or a pharmaceutically acceptable salt thereof, capsaicin, and hemp oil and methods of using the topical pharmaceutical compositions to treat neuropathic and nociceptive pain.

### CROSS REFERENCE TO RELATED APPLICATION

This application is a Non-Provisional application, which claims the benefit of, and priority to, U.S. Provisional Application No. 62/913,355 filed Oct. 10, 2019, which is incorporated herein by reference in its entirety.

### BACKGROUND

Peripheral neuropathy, a result of damage to nerves outside of the brain and spinal cord, is one of the most prevalent neurological conditions. There are numerous causes including, but not limited to, nerve damage associated with diabetes, HIV, infections, injury, and the administration of chemotherapeutic agents. Symptoms of peripheral neuropathy often include neuropathic pain.

The current treatment options available to patients with peripheral neuropathy and neuropathic pain, e.g., anticonvulsants, antidepressants, non-steroidal anti-inflammatory drugs, and opioids, often lack efficacy and/or are associated with adverse reactions.

Accordingly, there remains an unmet need to develop pharmaceutical compositions with the necessary efficacy and safety profiles required to successfully treat peripheral neuropathy and neuropathic pain.

### SUMMARY

In one aspect, provided herein are topical pharmaceutical compositions comprising lidocaine or a pharmaceutically acceptable salt thereof, capsaicin and hemp oil.

In various embodiments, the topical pharmaceutical composition comprises: (i) lidocaine or a pharmaceutically acceptable salt thereof; (ii) capsaicin; (iii) hemp oil; and (iv) a pharmaceutically acceptable carrier.

In certain embodiments, the topical pharmaceutical composition comprises from about 3% (w/w) to about 5% (w/w) lidocaine or a pharmaceutically acceptable salt thereof, wherein the weight percentages are based on the total weight of the topical pharmaceutical composition. In certain embodiments, the topical pharmaceutical composition comprises from about 1% (w/w) to about 2% (w/w) capsaicin, wherein the weight percentages are based on the total weight of the topical pharmaceutical composition. In certain embodiments, the topical pharmaceutical composition comprises from about 0.2% (w/w) to about 3% (w/w) hemp oil, wherein the weight percentages are based on the total weight of the topical pharmaceutical composition.

In various embodiments, the topical pharmaceutical composition comprises: (i) about 3% (w/w) to about 5% (w/w) lidocaine or a pharmaceutically acceptable salt thereof; (ii) about 1% (w/w) to about 2% (w/w) capsaicin; (iii) about 0.2% (w/w) to about 3% (w/w) hemp oil; and (iv) a pharmaceutically acceptable carrier, wherein the weight percentages are based on the total weight of the topical pharmaceutical composition.

In certain embodiments, the topical pharmaceutical composition further comprises N-palmitoylethanolamine. In some embodiments, the topical pharmaceutical composition comprises from about 1% (w/w) to about 3% (w/w) N-palmitoylethanolamine, wherein the weight percentages are based on the total weight of the topical pharmaceutical composition.

In certain embodiments, the topical pharmaceutical composition further comprises Acmella oleracea extract. In some embodiments, the topical pharmaceutical composition comprises from about 3% (w/w) to about 5% (w/w) Acmella oleracea extract, wherein the weight percentages are based on the total weight of the topical pharmaceutical composition.

In certain embodiments, the topical pharmaceutical composition further comprises methylsulfonylmethane. In some embodiments, the topical pharmaceutical composition comprises from about 8% (w/w) to about 12% (w/w) methylsulfonylmethane, wherein the weight percentages are based on the total weight of the topical pharmaceutical composition.

In certain embodiments, the topical pharmaceutical composition further comprises geranium oil. In some embodiments, the topical pharmaceutical composition comprises from about 1% (w/w) to about 3% (w/w) geranium oil, wherein the weight percentages are based on the total weight of the topical pharmaceutical composition.

In certain embodiments, the topical pharmaceutical composition further comprises lavender oil. In some embodiments, the topical pharmaceutical composition comprises from about 1% (w/w) to about 3% (w/w) lavender oil, wherein the weight percentages are based on the total weight of the topical pharmaceutical composition.

In certain embodiments, the topical pharmaceutical composition comprises from about 60% (w/w) to about 85% (w/w) of the pharmaceutically acceptable carrier, wherein the weight percentages are based on the total weight of the topical pharmaceutical composition. In some embodiments, the pharmaceutically acceptable carrier is an emulsified lotion base.

In certain embodiments, the pharmaceutically acceptable salt of lidocaine is a hydrochloride salt.

In various embodiments, the topical pharmaceutical composition comprises: (i) about 3% (w/w) to about 5% (w/w) lidocaine hydrochloride; (ii) about 1% (w/w) to about 2% (w/w) capsaicin; (iii) about 0.2% (w/w) to about 3% (w/w) hemp oil; (iv) about 1% (w/w) to about 3% (w/w) N-palmitoylethanolamine; (v) about 3% (w/w) to about 5% (w/w) Acmella oleracea extract; (vi) about 8% (w/w) to about 12% (w/w) methylsulfonylmethane; (vii) about 1% (w/w) to about 3% (w/w) geranium oil; (viii) about 1% (w/w) to about 3% (w/w) lavender oil; and (ix) about 60% (w/w) to about 85% (w/w) of an emulsified lotion base, wherein the weight percentages are based on the total weight of the topical pharmaceutical composition.

In various embodiments, the topical pharmaceutical composition comprises: (i) about 4% (w/w) lidocaine hydrochloride; (ii) about 1.5% (w/w) capsaicin; (iii) about 2.5% (w/w) hemp oil; (iv) about 2% (w/w) N-palmitoylethanolamine; (v) about 4% (w/w) Acmella oleracea extract; (vi) about 10% (w/w) methylsulfonylmethane; (vii) about 2% (w/w) geranium oil; (viii) about 2% (w/w) lavender oil; and (ix) about 72% (w/w) of an emulsified lotion base, wherein the weight percentages are based on the total weight of the topical pharmaceutical composition.

In various embodiments, the topical pharmaceutical composition comprises: (i) about 4% (w/w) lidocaine hydrochloride; (ii) about 1.5% (w/w) capsaicin; (iii) about 0.4% (w/w) hemp oil; (iv) about 2% (w/w) N-palmitoylethanolamine; (v) about 4% (w/w) Acmella oleracea extract; (vi) about 10% (w/w) methylsulfonylmethane; (vii) about 2% (w/w) geranium oil; (viii) about 2% (w/w) lavender oil; and (ix) about 74.1% (w/w) of an emulsified lotion base, wherein the weight percentages are based on the total weight of the topical pharmaceutical composition.

In certain embodiments, the hemp oil comprises one or more cannabinoids. In certain embodiments, the topical pharmaceutical composition comprises from about 0.69% (w/w) to about 0.79% (w/w) cannabidiol, wherein the weight percentage is based on the total weight of the topical pharmaceutical composition. In certain embodiments, the topical pharmaceutical composition comprises from about 0.02% (w/w) to about 0.04% (w/w) cannabichromene, wherein the weight percentage is based on the total weight of the topical pharmaceutical composition. In certain embodiments, the pharmaceutical composition comprises less than about 0.3% (w/w) Δ⁹-tetrahydrocannabinol, wherein the weight percentage is based on the total weight of the topical pharmaceutical composition.

In another aspect, provided herein is a method of treating neuropathic and nociceptive pain in a patient in need thereof, the method comprising administering to the patient an effective amount of a topical pharmaceutical composition described herein.

In another aspect, provided herein is a method of treating peripheral neuropathy in a patient in need thereof, the method comprising administering to the patient an effective amount of a topical pharmaceutical composition described herein.

### DETAILED DESCRIPTION

As generally described herein, the present disclosure provides topical pharmaceutical compositions comprising lidocaine or a pharmaceutically acceptable salt thereof, capsaicin and hemp oil, and methods of using the topical pharmaceutical compositions to treat medical disorders e.g., neuropathic pain, nociceptive pain and peripheral neuropathy.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a +10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

As used herein, “pharmaceutical composition” or “pharmaceutical formulation” refers to the combination of a therapeutically active agent with a pharmaceutically acceptable excipient, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or human, as appropriate; or means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present invention (e.g., lidocaine), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, Pa. (1975).

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, sachets, reconstitutable powders, dry powder inhalers and chewables.

As used herein, “administering” means oral administration, administration as a suppository, topical contact, intravenous administration, parenteral administration, intraperitoneal administration, intramuscular administration, intralesional administration, intrathecal administration, intracranial administration, intranasal administration or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The topical pharmaceutical compositions disclosed herein can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a compound refers to an amount sufficient to elicit the desired biological response, e.g., to treat neuropathic and nociceptive pain. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

As used herein, the term “Cannabis” refers to plants of the genus Cannabis, including Cannabis sativa, Cannabis indica, and Cannabis ruderalis.

As used herein, the terms “hemp oil”, “hemp seed oil”, or “hempseed oil” refer to a mixture of compounds obtained extracted from a hemp plant (e.g., a strain of the Cannabis sativa plant species). Such compounds include, but are not limited to, cannabinoids, terpenes, terpenoids, polyunsaturated fatty acids, and other compounds found in the hemp plant. In certain embodiments, hemp oil does not contain significant amounts of tetrahydrocannabinol. The exact composition of hemp oil will depend on the strain/variety of Cannabis plant, the efficiency and process of the extraction itself, and any additives that might be incorporated to alter the properties or improve administration of the hemp oil.

As used herein, the term “cannabinoid” refers to a chemical compound that shows direct or indirect activity at a cannabinoid receptor. There are two main cannabinoid receptors, CNR1 (also known as CB1) and CNR2 (also known as CB2). Other receptors that research indicates have cannabinoid activity include the GPR55, GPR18, and TRPV1 receptors. The term “phytocannabinoid” refers to cannabinoids that occur in a plant species or are derived from cannabinoids occurring in a plant species. Examples of cannabinoids include, but are not limited to, tetrahydrocannabinol (THC), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

As used herein, the term “acidic cannabinoid” refers to a cannabinoid having one or more carboxylic acid functional groups. Examples of acidic cannabinoids include, but are not limited to, tetrahydrocannabinolic acid (THCA), cannabidiolic acid (CBDA), and cannabichromenic acid (CBC). Acidic cannabinoids are frequently the predominant cannabinoids found in raw (unprocessed) Cannabis plant material.

As used herein, the term “neutral cannabinoid” refers to a cannabinoid without carboxylic acid functional groups. Examples of neutral cannabinoids include, but are not limited to, THC, CBD, CBG, CBC, and CBN.

### Topical Pharmaceutical Compositions

As described herein, in one aspect, the disclosure provides pharmaceutical compositions lidocaine or a pharmaceutically acceptable salt thereof, capsaicin and hemp oil.

In various embodiments, a topical pharmaceutical composition generally comprises:

(i) lidocaine or a pharmaceutically acceptable salt thereof,

(ii) capsaicin;

(iii) hemp oil; and

(iv) a pharmaceutically acceptable carrier.

In certain embodiments, the topical pharmaceutical composition comprises from about 3% (w/w) to about 5% (w/w), about 3.5% (w/w) to about 5% (w/w), about 4% (w/w) to about 50% (w/w), about 4.50% (w/w) to about 5% (w/w), about 30% (w/w) to about 4.50% (w/w), about 3% (w/w) to about 4% (w/w), about 3% (w/w) to about 3.5% (w/w), about 3.5% (w/w) to about 4.5% (w/w), about 3.5% (w/w) to about 4% (w/w), or about 4% (w/w) to about 4.5% (w/w) lidocaine or a pharmaceutically acceptable salt thereof, wherein the weight percentages are based on the total weight of the topical pharmaceutical composition. In some embodiments, the topical pharmaceutical composition comprises from about 3% (w/w) to about 5% (w/w) lidocaine or a pharmaceutically acceptable salt thereof, wherein the weight percentages are based on the total weight of the topical pharmaceutical composition.

In certain embodiments, the topical pharmaceutical composition comprises about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), or about 7% (w/w) lidocaine or a pharmaceutically acceptable salt thereof, wherein the weight percentages are based on the total weight of the topical pharmaceutical composition. In some embodiments, the topical pharmaceutical composition comprises about 4% (w/w) lidocaine or a pharmaceutically acceptable salt thereof, wherein the weight percentages are based on the total weight of the topical pharmaceutical composition.

In certain embodiments, the topical pharmaceutical composition comprises from about 1% (w/w) to about 2% (w/w), about 1.25% (w/w) to about 2% (w/w), about 1.5% (w/w) to about 2% (w/w), about 1.75% (w/w) to about 2% (w/w), about 1% (w/w) to about 1.75% (w/w), about 1% (w/w) to about 1.5% (w/w), about 1% (w/w) to about 1.25% (w/w), about 1.25% (w/w) to about 1.75% (w/w), about 1.25% (w/w) to about 1.50% (w/w), or about 1.50% (w/w) to about 1.75% (w/w) capsaicin, wherein the weight percentages are based on the total weight of the topical pharmaceutical composition. In some embodiments, the topical pharmaceutical composition comprises from about 1% (w/w) to about 2% (w/w) capsaicin, wherein the weight percentages are based on the total weight of the topical pharmaceutical composition.

In certain embodiments, the topical pharmaceutical composition comprises about 0.5% (w/w), about 0.75% (w/w), about 1% (w/w), about 1.25% (w/w), about 1.5% (w/w), about 1.75% (w/w), about 2% (w/w), about 2.25% (w/w), or about 2.5% (w/w) capsaicin, wherein the weight percentages are based on the total weight of the topical pharmaceutical composition. In some embodiments, the topical pharmaceutical composition comprises about 1.5% (w/w) capsaicin, wherein the weight percentages are based on the total weight of the topical pharmaceutical composition.

In certain embodiments, the topical pharmaceutical composition comprises from about 0.2% (w/w) to about 3% (w/w), about 0.5% (w/w) to about 3% (w/w), about 1% (w/w) to about 3% (w/w), about 1.5% (w/w) to about 3% (w/w), about 2% (w/w) to about 3% (w/w), about 2.5% (w/w) to about 3% (w/w), about 0.2% (w/w) to about 2.5% (w/w), about 0.2% (w/w) to about 2% (w/w), about 0.2% (w/w) to about 1.5% (w/w), about 0.2% (w/w) to about 1% (w/w), about 0.2% (w/w) to about 0.50% (w/w), about 0.50% (w/w) to about 2.5% (w/w), about 0.50% (w/w) to about 2% (w/w), about 0.50% (w/w) to about 1.50% (w/w), about 0.50% (w/w) to about 1% (w/w), about 1% (w/w) to about 2.5% (w/w), about 1% (w/w) to about 2% (w/w), about 1% (w/w) to about 1.50% (w/w), about 1.50% (w/w) to about 2.5% (w/w), about 1.50% (w/w) to about 2% (w/w), or about 2% (w/w) to about 2.5% (w/w) hemp oil, wherein the weight percentages are based on the total weight of the topical pharmaceutical composition. In some embodiments, the topical pharmaceutical composition comprises from about 0.2% (w/w) to about 3% (w/w) hemp oil, wherein the weight percentages are based on the total weight of the topical pharmaceutical composition.

In certain embodiments, the topical pharmaceutical composition comprises about 0.2% (w/w), about 0.3% (w/w), about 0.4% (w/w), about 0.5% (w/w), about 1% (w/w), about 1.5% (w/w), about 2% (w/w), about 2.5% (w/w), or about 3% (w/w) hemp oil, wherein the weight percentages are based on the total weight of the topical pharmaceutical composition. In some embodiments, the topical pharmaceutical composition comprises about 0.4% (w/w) hemp oil, wherein the weight percentages are based on the total weight of the topical pharmaceutical composition. In some embodiments, the topical pharmaceutical composition comprises about 2.5% (w/w) hemp oil, wherein the weight percentages are based on the total weight of the topical pharmaceutical composition.

In various embodiments, the topical pharmaceutical composition comprises:

(i) about 3% (w/w) to about 5% (w/w) lidocaine or a pharmaceutically acceptable salt thereof;

(ii) about 1% (w/w) to about 2% (w/w) capsaicin;

(iii) about 0.2% (w/w) to about 3% (w/w) hemp oil; and

(iv) a pharmaceutically acceptable carrier,

wherein the weight percentages are based on the total weight of the topical pharmaceutical composition.

In certain embodiments, the topical pharmaceutical composition further comprises N-palmitoylethanolamine.

In certain embodiments, the topical pharmaceutical composition comprises from about 10% (w/w) to about 30% (w/w), about 1.5% (w/w) to about 30% (w/w), about 2% (w/w) to about 3% (w/w), about 2.5% (w/w) to about 3% (w/w), about 1% (w/w) to about 2.5% (w/w), about 1% (w/w) to about 2% (w/w), about 1% (w/w) to about 1.5% (w/w), about 1.5% (w/w) to about 2.5% (w/w), about 1.5% (w/w) to about 2% (w/w), or about 2% (w/w) to about 2.5% (w/w) N-palmitoylethanolamine, wherein the weight percentages are based on the total weight of the topical pharmaceutical composition. In some embodiments, the topical pharmaceutical composition comprises from about 1% (w/w) to about 3% (w/w) N-palmitoylethanolamine, wherein the weight percentages are based on the total weight of the topical pharmaceutical composition.

In certain embodiments, the topical pharmaceutical composition comprises about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), or about 5% (w/w) N-palmitoylethanolamine, wherein the weight percentages are based on the total weight of the topical pharmaceutical composition. In some embodiments, the topical pharmaceutical composition comprises about 2% (w/w) N-palmitoylethanolamine, wherein the weight percentages are based on the total weight of the topical pharmaceutical composition.

In certain embodiments, the topical pharmaceutical composition further comprises Acmella oleracea extract. In certain embodiments, the Acmella oleracea extract comprises dried Acmella oleracea plant material, a solvent, or a combination thereof. In some embodiments, the ratio of the weight of the dried Acmella oleracea plant material to the volume of the solvent is from about 1:3. In some embodiments, the solvent comprises glycerin.

In certain embodiments, the topical pharmaceutical composition comprises from about 3% (w/w) to about 5% (w/w), about 3.5% (w/w) to about 5% (w/w), about 4% (w/w) to about 50% (w/w), about 4.5% (w/w) to about 50% (w/w), about 30% (w/w) to about 4.5% (w/w), about 3% (w/w) to about 4% (w/w), about 3% (w/w) to about 3.5% (w/w), about 3.5% (w/w) to about 4.5% (w/w), about 3.5% (w/w) to about 4% (w/w), or about 4% (w/w) to about 4.5% (w/w) Acmella oleracea extract, wherein the weight percentages are based on the total weight of the topical pharmaceutical composition. In some embodiments, the topical pharmaceutical composition comprises from about 3% (w/w) to about 5% (w/w) Acmella oleracea extract, wherein the weight percentages are based on the total weight of the topical pharmaceutical composition.

In certain embodiments, the topical pharmaceutical composition comprises about 1% (w/w), about 2% (w/w), about 3% (w/w), about 4% (w/w), about 5% (w/w), about 6% (w/w), about 7% (w/w), about 8% (w/w), about 9% (w/w), or about 10% (w/w) Acmella oleracea extract, wherein the weight percentages are based on the total weight of the topical pharmaceutical composition. In some embodiments, the topical pharmaceutical composition comprises about 4% (w/w) Acmella oleracea extract, wherein the weight percentages are based on the total weight of the topical pharmaceutical composition.

In certain embodiments, the topical pharmaceutical composition further comprises methylsulfonylmethane.

In certain embodiments, the topical pharmaceutical composition comprises from about 8% (w/w) to about 12% (w/w), about 9% (w/w) to about 12% (w/w), about 10% (w/w) to about 12% (w/w), about 11% (w/w) to about 12% (w/w), about 8% (w/w) to about 11% (w/w), about 8% (w/w) to about 10% (w/w), about 8% (w/w) to about 9% (w/w), about 9% (w/w) to about 11% (w/w), about 9% (w/w) to about 10% (w/w), or about 10% (w/w) to about 11% (w/w) methylsulfonylmethane, wherein the weight percentages are based on the total weight of the topical pharmaceutical composition. In some embodiments, the topical pharmaceutical composition comprises from about 8% (w/w) to about 12% (w/w) methylsulfonylmethane, wherein the weight percentages are based on the total weight of the topical pharmaceutical composition.

In certain embodiments, the topical pharmaceutical composition comprises about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), or about 15% (w/w) methylsulfonylmethane, wherein the weight percentages are based on the total weight of the topical pharmaceutical composition. In some embodiments, the topical pharmaceutical composition comprises about 10% (w/w) methylsulfonylmethane, wherein the weight percentages are based on the total weight of the topical pharmaceutical composition.

In certain embodiments, the topical pharmaceutical composition further comprises geranium oil. In certain embodiments, the geranium oil is derived from the Pelargonium graveolens plant using steam distillation.

In certain embodiments, the topical pharmaceutical composition comprises from about 10% (w/w) to about 30% (w/w), about 1.5% (w/w) to about 30% (w/w), about 2% (w/w) to about 3% (w/w), about 2.5% (w/w) to about 3% (w/w), about 1% (w/w) to about 2.5% (w/w), about 1% (w/w) to about 2% (w/w), about 1% (w/w) to about 1.5% (w/w), about 1.5% (w/w) to about 2.5% (w/w), about 1.5% (w/w) to about 2% (w/w), or about 2% (w/w) to about 2.5% (w/w) geranium oil, wherein the weight percentages are based on the total weight of the topical pharmaceutical composition. In some embodiments, the topical pharmaceutical composition comprises from about 1% (w/w) to about 3% (w/w) geranium oil, wherein the weight percentages are based on the total weight of the topical pharmaceutical composition.

In certain embodiments, the topical pharmaceutical composition comprises about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), or about 5% (w/w) geranium oil, wherein the weight percentages are based on the total weight of the topical pharmaceutical composition. In some embodiments, the topical pharmaceutical composition comprises about 2% (w/w) geranium oil, wherein the weight percentages are based on the total weight of the topical pharmaceutical composition.

In certain embodiments, the topical pharmaceutical composition further comprises lavender oil. In certain embodiments, the lavender oil is derived from the Lavandula angustifolia plant using steam distillation.

In certain embodiments, the topical pharmaceutical composition comprises from about 1% (w/w) to about 30% (w/w), about 1.50% (w/w) to about 30% (w/w), about 2% (w/w) to about 3% (w/w), about 2.5% (w/w) to about 3% (w/w), about 1% (w/w) to about 2.5% (w/w), about 1% (w/w) to about 2% (w/w), about 1% (w/w) to about 1.50% (w/w), about 1.50% (w/w) to about 2.5% (w/w), about 1.5% (w/w) to about 2% (w/w), or about 2% (w/w) to about 2.5% (w/w) lavender oil, wherein the weight percentages are based on the total weight of the topical pharmaceutical composition. In some embodiments, the topical pharmaceutical composition comprises from about 1% (w/w) to about 3% (w/w) lavender oil, wherein the weight percentages are based on the total weight of the topical pharmaceutical composition.

In certain embodiments, the topical pharmaceutical composition comprises about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), or about 5% (w/w) lavender oil, wherein the weight percentages are based on the total weight of the topical pharmaceutical composition. In some embodiments, the topical pharmaceutical composition comprises about 2% (w/w) lavender oil, wherein the weight percentages are based on the total weight of the topical pharmaceutical composition.

In certain embodiments, the topical pharmaceutical composition comprises from about 60% (w/w) to about 85% (w/w), about 65% (w/w) to about 85% (w/w), about 70% (w/w) to about 85% (w/w), about 75% (w/w) to about 85% (w/w), about 80% (w/w) to about 85% (w/w), about 60% (w/w) to about 80% (w/w), about 60% (w/w) to about 75% (w/w), about 60% (w/w) to about 70% (w/w), about 60% (w/w) to about 65% (w/w), about 65% (w/w) to about 80% (w/w), about 65% (w/w) to about 75% (w/w), about 65% (w/w) to about 70% (w/w), about 70% (w/w) to about 80% (w/w), about 70% (w/w) to about 75% (w/w), or about 75% (w/w) to about 80% (w/w) of the pharmaceutically acceptable carrier, wherein the weight percentages are based on the total weight of the topical pharmaceutical composition. In some embodiments, the topical pharmaceutical composition comprises from about 60% (w/w) to about 85% (w/w) of the pharmaceutically acceptable carrier, wherein the weight percentages are based on the total weight of the topical pharmaceutical composition.

In certain embodiments, the topical pharmaceutical composition comprises about 60% (w/w), about 65% (w/w), about 70% (w/w), about 75% (w/w), about 80% (w/w), or about 85% (w/w) of the pharmaceutically acceptable carrier, wherein the weight percentages are based on the total weight of the topical pharmaceutical composition. In certain embodiments, the topical pharmaceutical composition comprises about 70% (w/w), about 71% (w/w), about 72% (w/w), about 73% (w/w), about 74% (w/w), about 75% (w/w), about 76% (w/w), about 77% (w/w), about 78% (w/w), about 79% (w/w), or about 80% (w/w) of the pharmaceutically acceptable carrier, wherein the weight percentages are based on the total weight of the topical pharmaceutical composition. In some embodiments, the topical pharmaceutical composition comprises about 72% (w/w) of the pharmaceutically acceptable carrier, wherein the weight percentages are based on the total weight of the topical pharmaceutical composition. In some embodiments, the topical pharmaceutical composition comprises about 74.1% (w/w) of the pharmaceutically acceptable carrier, wherein the weight percentages are based on the total weight of the topical pharmaceutical composition.

In certain embodiments, the pharmaceutically acceptable carrier comprises an olive oil. In certain embodiments, the pharmaceutically acceptable carrier comprises a medium chain triglyceride (MCT) oil.

In certain embodiments, the pharmaceutically acceptable carrier is an emulsion. In certain embodiments, the pharmaceutically acceptable carrier is an oil-in-water emulsion. In certain embodiments, the pharmaceutically acceptable carrier is a water-in-oil emulsion.

In certain embodiments, the pharmaceutically acceptable carrier is an emulsified lotion base. In certain embodiments, the emulsified lotion base comprises one or more pharmaceutically acceptable excipients selected from the group consisting of aloe vera juice, shea butter, an emulsifying wax, vegetable glycerin, stearic acid, silver dihydrogen citrate, citric acid, and combinations thereof.

In certain embodiments, the pharmaceutically acceptable salt of lidocaine is a hydrochloride salt.

In various embodiments, the topical pharmaceutical composition comprises:

(i) about 3% (w/w) to about 5% (w/w) lidocaine hydrochloride;

(ii) about 1% (w/w) to about 2% (w/w) capsaicin;

(iii) about 0.2% (w/w) to about 3% (w/w) hemp oil;

(iv) about 1% (w/w) to about 3% (w/w) N-palmitoylethanolamine;

(v) about 3% (w/w) to about 5% (w/w) Acmella oleracea extract;

(vi) about 8% (w/w) to about 12% (w/w) methylsulfonylmethane;

(vii) about 1% (w/w) to about 3% (w/w) geranium oil;

(viii) about 1% (w/w) to about 3% (w/w) lavender oil; and

(ix) about 60% (w/w) to about 85% (w/w) of an emulsified lotion base,

wherein the weight percentages are based on the total weight of the topical pharmaceutical composition.

In various embodiments, the topical pharmaceutical composition comprises:

(i) about 4% (w/w) lidocaine hydrochloride;

(ii) about 1.5% (w/w) capsaicin;

(iii) about 2.5% (w/w) hemp oil;

(iv) about 2% (w/w) N-palmitoylethanolamine;

(v) about 4% (w/w) Acmella oleracea extract;

(vi) about 10% (w/w) methylsulfonylmethane;

(vii) about 2% (w/w) geranium oil;

(viii) about 2% (w/w) lavender oil; and

(ix) about 72% (w/w) of an emulsified lotion base,

wherein the weight percentages are based on the total weight of the topical pharmaceutical composition.

In various embodiments, the topical pharmaceutical composition comprises:

(i) about 4% (w/w) lidocaine hydrochloride;

(ii) about 1.5% (w/w) capsaicin;

(iii) about 0.4% (w/w) hemp oil;

(iv) about 2% (w/w) N-palmitoylethanolamine;

(v) about 4% (w/w) Acmella oleracea extract;

(vi) about 10% (w/w) methylsulfonylmethane;

(vii) about 2% (w/w) geranium oil;

(viii) about 2% (w/w) lavender oil; and

(ix) about 74.1% (w/w) of an emulsified lotion base,

wherein the weight percentages are based on the total weight of the topical pharmaceutical composition.

In certain embodiments, the hemp oil described herein is extracted from a hemp plant using a supercritical carbon dioxide extraction process. In certain embodiments, the hemp plant is a strain of Cannabis sativa.

In certain embodiments, the hemp oil comprises one or more cannabinoids.

In certain embodiments, the topical pharmaceutical composition comprises from about 0.5% (w/w) to about 1% (w/w), about 0.6% (w/w) to about 1% (w/w), about 0.7% (w/w) to about 1% (w/w), about 0.8% (w/w) to about 1% (w/w), about 0.9% (w/w) to about 1% (w/w), about 0.5% (w/w) to about 0.9% (w/w), about 0.5% (w/w) to about 0.8% (w/w), about 0.5% (w/w) to about 0.7% (w/w), about 0.5% (w/w) to about 0.6% (w/w), about 0.6% (w/w) to about 0.9% (w/w), about 0.6% (w/w) to about 0.8% (w/w), about 0.6% (w/w) to about 0.7% (w/w), about 0.7% (w/w) to about 0.9% (w/w), about 0.7% (w/w) to about 0.8% (w/w), or about 0.8% (w/w) to about 0.9% (w/w) of the one or more cannabinoids, wherein the weight percentages are based on the total weight of the topical pharmaceutical composition. In some embodiments, the topical pharmaceutical composition comprises from about 0.7% (w/w) to about 0.9% (w/w) of the one or more cannabinoids, wherein the weight percentages are based on the total weight of the topical pharmaceutical composition.

In certain embodiments, the topical pharmaceutical composition comprises about 0.3% (w/w), about 0.4% (w/w), about 0.5% (w/w), about 0.6% (w/w), about 0.7% (w/w), about 0.8% (w/w), about 0.9% (w/w), or about 1% (w/w) of the one or more cannabinoids, wherein the weight percentages are based on the total weight of the topical pharmaceutical composition. In some embodiments, the topical pharmaceutical composition comprises about 0.8% (w/w) of the one or more cannabinoids, wherein the weight percentages are based on the total weight of the topical pharmaceutical composition.

In certain embodiments, the topical pharmaceutical composition comprises about 0.75% (w/w), about 0.76% (w/w), about 0.77% (w/w), about 0.78% (w/w), about 0.79% (w/w), about 0.8% (w/w), about 0.81% (w/w), about 0.82% (w/w), about 0.83% (w/w), about 0.84% (w/w), or about 0.85% (w/w) of the one or more cannabinoids, wherein the weight percentages are based on the total weight of the topical pharmaceutical composition. In some embodiments, the topical pharmaceutical composition comprises about 0.75% (w/w) of the one or more cannabinoids, wherein the weight percentages are based on the total weight of the topical pharmaceutical composition. In some embodiments, the topical pharmaceutical composition comprises about 0.82% (w/w) of the one or more cannabinoids, wherein the weight percentages are based on the total weight of the topical pharmaceutical composition.

In certain embodiments, the one or more cannabinoids comprise one or more neutral cannabinoids. In certain embodiments, the one or more neutral cannabinoids is selected from the group comprising cannabigerol (CBG); cannabigerol monomethyl ether; cannabigerovarin; cannabichromene (CBC); (±)-cannabichromene; (±)-cannabichromevarin; (−)-cannabidiol (CBD); cannabidiol monomethyl ether; cannabidiol-C₄; (−)-cannabidivarin; cannabidiorcol; cannabinodiol (CBND); cannabinodivarin; Δ⁸-tetrahydrocannabinol (Δ⁸-THC); Δ⁹-tetrahydrocannabinol (Δ⁹-THC); Δ⁹-tetrahydrocannabinol-C₄; tetrahydrocannabivarin (THCV); Δ⁹-tetrahydro-cannabiorcol; (−)-Δ⁹-trans-(6aR,10aR)-Δ⁹-tetrahydrocannabinol; (−)-(6aS,10aR)-Δ⁹-tetrahydrocannabinol; cannabinol (CBN); cannabinol-C₄; cannabivarin; cannabinol-C₂; cannabiorcol; cannabinol methyl ether; (±)-cannabitriol (CBT); (−)-(9R,10R)-trans-10-O-ethyl-cannabitriol; (±)-(9R,10R/9S,10S)-cannabitriol-C₃; cannabielsoin (CBE); (5aS,6S,9R,9aR)-cannabielsoin; (5aS,6S,9R,9aR)—C₃-cannabielsoin; cannabiglendol-C₃; dehydrocannabifuran; cannabifuran; an isocannabinoid; (−)-Δ⁷-trans-(1R,3R,6R)-isotetrahydrocannabinol; (±)-Δ⁷-1,2-cis-(1R,3R,6S)-isotetrahydrocannabivarin; (±)-Δ⁷-1,2-cis-(1S,3S,6R)-isotetrahydrocannabivarin; (−)-Δ⁷-trans-(1R,3R,6R)-isotetrahydrocannabivarin; cannabicyclol (CBL); (±)-(1aS,3aR,8bR,8cR)-cannabicyclol CBL-C₅; (±)-(1aS,3aR,8bR,8cR)-cannabicyclovarin; cannabicitran (CBT); cannabichromanone (CBCN); cannabichromanone-C₃; cannabicoumaronone; or combinations thereof. In some embodiments, the one or more neutral cannabinoids is selected from the group consisting of CBC, CBD, Δ⁹-THC, and combinations thereof.

In certain embodiments, the one or more cannabinoids further comprises one or more acidic cannabinoids. In certain embodiments, the one or more acidic cannabinoids is selected from the group comprising cannabigerolic acid A; cannabigerolic acid A monomethyl ether; cannabigerovarinic acid A; (±)-cannabichromenic acid A; (±)-cannabichromevarinic acid A; cannabidiolic acid (CBDA); cannabidivarinic acid; Δ⁹-tetrahydrocannabinolic acid A; Δ⁹-tetrahydrocannabinolic acid B; Δ⁹-tetrahydrocannabinolic acid-C₄ A; Δ⁹-tetrahydrocannabinolic acid-C₄ B; Δ⁹-tetrahydro-cannabivarinic acid A; Δ⁹-tetrahydrocannabiorcolic acid A; Δ⁹-tetrahydrocannabiorcolic acid B; (−)-Δ⁹-trans-(6aR,10aR)-tetrahydrocannabinolic acid A; cannabinolic acid A; (5aS,6S,9R,9aR)-cannabielsoic acid A; (5aS,6S,9R,9aR)-cannabielsoic acid B; (5aS,6S,9R,9aR)—C₃-cannabielsoic acid B; (±)-(1aS,3aR,8bR,8cR)-cannabicyclolic acid A; or combinations thereof.

In certain embodiments, the topical pharmaceutical composition comprises from about 0.01% (w/w) to about 0.05% (w/w), about 0.02% (w/w) to about 0.05% (w/w), about 0.03% (w/w) to about 0.050% (w/w), about 0.04% (w/w) to about 0.050% (w/w), about 0.01% (w/w) to about 0.04% (w/w), about 0.01% (w/w) to about 0.03% (w/w), about 0.01% (w/w) to about 0.02% (w/w), about 0.02% (w/w) to about 0.04% (w/w), about 0.02% (w/w) to about 0.03% (w/w), or about 0.03% (w/w) to about 0.04% (w/w) CBC, wherein the weight percentage is based on the total weight of the topical pharmaceutical composition. In some embodiments, the topical pharmaceutical composition comprises from about 0.02% (w/w) to about 0.04% (w/w) CBC, wherein the weight percentage is based on the total weight of the topical pharmaceutical composition.

In certain embodiments, the topical pharmaceutical composition comprises about 0.01% (w/w), about 0.02% (w/w), about 0.03% (w/w), about 0.04% (w/w), or about 0.05% (w/w) CBC, wherein the weight percentage is based on the total weight of the topical pharmaceutical composition. In some embodiments, the topical pharmaceutical composition comprises about 0.03% (w/w) CBC, wherein the weight percentage is based on the total weight of the topical pharmaceutical composition.

In certain embodiments, the topical pharmaceutical composition comprises from about 0.65% (w/w) to about 0.85% (w/w), about 0.67% (w/w) to about 0.85% (w/w), about 0.69% (w/w) to about 0.85% (w/w), about 0.71% (w/w) to about 0.85% (w/w), about 0.73% (w/w) to about 0.85% (w/w), about 0.75% (w/w) to about 0.85% (w/w), about 0.77% (w/w) to about 0.85% (w/w), about 0.79% (w/w) to about 0.81% (w/w), about 0.81% (w/w) to about 0.85% (w/w), about 0.83% (w/w) to about 0.85% (w/w), about 0.65% (w/w) to about 0.83% (w/w), about 0.65% (w/w) to about 0.81% (w/w), about 0.65% (w/w) to about 0.79% (w/w), about 0.65% (w/w) to about 0.77% (w/w), about 0.65% (w/w) to about 0.75% (w/w), about 0.65% (w/w) to about 0.73% (w/w), about 0.65% (w/w) to about 0.71% (w/w), about 0.65% (w/w) to about 0.69% (w/w), about 0.65% (w/w) to about 0.67% (w/w), about 0.67% (w/w) to about 0.83% (w/w), about 0.67% (w/w) to about 0.81% (w/w), about 0.67% (w/w) to about 0.79% (w/w), about 0.67% (w/w) to about 0.77% (w/w), about 0.67% (w/w) to about 0.75% (w/w), about 0.67% (w/w) to about 0.73% (w/w), about 0.67% (w/w) to about 0.71% (w/w), about 0.67% (w/w) to about 0.69% (w/w), about 0.69% (w/w) to about 0.83% (w/w), about 0.69% (w/w) to about 0.81% (w/w), about 0.69% (w/w) to about 0.79% (w/w), about 0.69% (w/w) to about 0.77% (w/w), about 0.69% (w/w) to about 0.75% (w/w), about 0.69% (w/w) to about 0.73% (w/w), about 0.69% (w/w) to about 0.71% (w/w), about 0.71% (w/w) to about 0.83% (w/w), about 0.71% (w/w) to about 0.81% (w/w), about 0.71% (w/w) to about 0.79% (w/w), about 0.71% (w/w) to about 0.77% (w/w), about 0.71% (w/w) to about 0.75% (w/w), about 0.71% (w/w) to about 0.73% (w/w), about 0.73% (w/w) to about 0.83% (w/w), about 0.73% (w/w) to about 0.81% (w/w), about 0.73% (w/w) to about 0.79% (w/w), about 0.73% (w/w) to about 0.77% (w/w), about 0.73% (w/w) to about 0.75% (w/w), about 0.75% (w/w) to about 0.83% (w/w), about 0.75% (w/w) to about 0.81% (w/w), about 0.75% (w/w) to about 0.79% (w/w), about 0.75% (w/w) to about 0.77% (w/w), about 0.77% (w/w) to about 0.83% (w/w), about 0.77% (w/w) to about 0.81% (w/w), about 0.77% (w/w) to about 0.79% (w/w), about 0.79% (w/w) to about 0.83% (w/w), about 0.79% (w/w) to about 0.81% (w/w), or about 0.81% (w/w) to about 0.83% (w/w) CBD, wherein the weight percentage is based on the total weight of the topical pharmaceutical composition. In some embodiments, the topical pharmaceutical composition comprises from about 0.69% (w/w) to about 0.79% (w/w) CBD, wherein the weight percentage is based on the total weight of the topical pharmaceutical composition.

In certain embodiments, the topical pharmaceutical composition comprises about 0.65% (w/w), about 0.66% (w/w), about 0.67% (w/w), about 0.68% (w/w), about 0.69% (w/w), about 0.7% (w/w), about 0.71% (w/w), about 0.72% (w/w), about 0.73% (w/w), about 0.74% (w/w), about 0.75% (w/w), about 0.76% (w/w), about 0.77% (w/w), about 0.78% (w/w), about 0.79% (w/w), or about 0.8% (w/w) CBD, wherein the weight percentages are based on the total weight of the topical pharmaceutical composition. In some embodiments, the topical pharmaceutical composition comprises about 0.7% (w/w) CBD, wherein the weight percentages are based on the total weight of the topical pharmaceutical composition. In some embodiments, the topical pharmaceutical composition comprises about 0.77% (w/w) CBD, wherein the weight percentages are based on the total weight of the topical pharmaceutical composition.

In certain embodiments, the topical pharmaceutical composition comprises less than about 0.02% (w/w), less than about 0.04% (w/w), less than about 0.06% (w/w), less than about 0.08% (w/w), less than about 0.1% (w/w), less than about 0.2% (w/w), less than about 0.3% (w/w), less than about 0.4% (w/w), less than about 0.5% (w/w), or less than about 0.6% (w/w) Δ⁹-THC, wherein the weight percentages are based on the total weight of the topical pharmaceutical composition. In some embodiments, the topical pharmaceutical composition comprises less than about 0.02% (w/w) Δ⁹-THC, wherein the weight percentage is based on the total weight of the topical pharmaceutical composition. In some embodiments, the topical pharmaceutical composition comprises less than about 0.3% (w/w) Δ⁹-THC, wherein the weight percentage is based on the total weight of the topical pharmaceutical composition.

In certain embodiments, the topical pharmaceutical composition comprises about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), or about 0.1% (w/w) Δ⁹-THC, wherein the weight percentage is based on the total weight of the topical pharmaceutical composition. In some embodiments, the topical pharmaceutical composition comprises about 0.02% (w/w) Δ⁹-THC, wherein the weight percentage is based on the total weight of the topical pharmaceutical composition.

In certain embodiments, the hemp oil comprises one or more terpenoids. In certain embodiments, the terpenoid is selected from the group comprising β-caryophyllene [(1R,4E,9S)-4,11,11-trimethyl-8-methylene-bicyclo(7.2.0)undec-4-ene]; β-caryophyllene oxide; citronellol [3,7-dimethyl-6-octen-1-ol]; α-eudesmol [2-[(2R,4aR)-4a,8-dimethyl-2,3,4,5,6,8a-hexahydro-1H-naphthalen-2-yl]propan-2-ol]; β-eudesmol [2-[(2R,4aR,8aS)-4a-methyl-8-methylidene-1,2,3,4,5,6,7,8a-octahydronaphthalen-2-yl]propan-2-ol]; γ-eudesmol [2-[(2R,4aR)-4a,8-dimethyl-2,3,4,5,6,7-hexahydro-1H-naphthalen-2-yl]propan-2-ol]; geraniol [(2E)-3,7-dimethylocta-2,6-dien-1-ol]; guaiol [2-[(3S,5R,8S)-3,8-dimethyl-1,2,3,4,5,6,7,8-octahydroazulen-5-yl]propan-2-ol]; α-humulene [(1E,4E,8E)-2,6,6,9-tetramethylcycloundeca-1,4,8-triene]; β-humulene [(1E,5E)-1,4,4-trimethyl-8-methylidenecycloundeca-1,5-diene]; γ-humulene [(1Z,6E)-1,8,8-trimethyl-5-methylidenecycloundeca-1,6-diene]; D-limonene [(4R)-1-methyl-4-prop-1-en-2-ylcyclohexene]; L-limonene [(4S)-1-methyl-4-prop-1-en-2-ylcyclohexene]; (−)-linalool [(3R)-3,7-dimethylocta-1,6-dien-3-ol]; (+)-linalool [(3S)-3,7-dimethylocta-1,6-dien-3-ol]; α-myrcene [2-methyl-6-methylideneocta-1,7-diene]; β-myrcene [7-methyl-3-methylideneocta-1,6-diene]; nerol [(2Z)-3,7-dimethylocta-2,6-dien-1-ol]; cis-nerolidol [(6Z)-3,7,11-trimethyldodeca-1,6,10-trien-3-ol]; trans-nerolidol [(6E)-3,7,11-trimethyldodeca-1,6,10-trien-3-ol]; α-ocimene [(3E)-3,7-dimethylocta-1,3,7-triene]; β-ocimene [(3E)-3,7-dimethylocta-1,3,6-triene]; p-cymene [1-methyl-4-(1-methylethyl)benzene]; α-phellandrene [2-methyl-5-propan-2-ylcyclohexa-1,3-diene]; β-phellandrene [3-methylidene-6-propan-2-ylcyclohexene]; cis-phytol [(Z,7R,11R)-3,7,11,15-tetramethylhexadec-2-en-1-ol]; trans-phytol [(E,7R,11R)-3,7,11,15-tetramethylhexadec-2-en-1-ol]; (−)-α-pinene [(1S,5S)-4,6,6-trimethylbicyclo[3.1.1]hept-3-ene]; (−)-β-pinene [(1S,5S)-6,6-dimethyl-4-methylidenebicyclo[3.1.1]heptane]; (+)-α-pinene [(1R,5R)-4,6,6-trimethylbicyclo[3.1.1]hept-3-ene]; (+)-β-pinene [(1R,5R)-6,6-dimethyl-4-methydenebicyclo[3.1.1]heptane]; (−)-pulegone [(5S)-5-methyl-2-propan-2-ylidenecyclohexan-1-one]; (+)-pulegone [(5R)-5-methyl-2-propan-2-ylidenecyclohexan-1-one]; α-terpinene [1-methyl-4-propan-2-ylcyclohexa-1,3-diene]; S-terpinene [5-methyl-2-propan-2-ylcyclohexa-1,3-diene]; γ-terpinene [1-methyl-4-propan-2-ylcyclohexa-1,4-diene]; α-terpineol [2-(4-methylcyclohex-3-en-1-yl)propan-2-ol]; γ-terpineol [1-methyl-4-propan-2-ylidenecyclohexan-1-ol]; (+)-valencene [(3R,4aS,5R)-4a,5-dimethyl-3-prop-1-en-2-yl-2,3,4,5,6,7-hexahydro-1H-naphthalene].

In certain embodiments, the hemp oil comprises one or more terpenes. In certain embodiments, the terpene is selected from the group comprising alpha-pinene, beta-pinene, delta-3 carene, p-cymene, myrcene, limonene, beta-ocimene, terpinolene, beta-caryophyllene, bergamotene, farnesene, alpha-humulene, fenchene, fenchol, eucalptol, borneol, alpha-terpineol, pulegone, linalool or combinations thereof.

In certain embodiments, the concentration of arsenic in the hemp oil is less than about 2 μg/kg, less than about 3 μg/kg, less than about 4 μg/kg, less than about 5 μg/kg, less than about 6 μg/kg, or less than about 7 μg/kg. In some embodiments, the concentration of arsenic in the hemp oil is less than about 4 μg/kg.

In certain embodiments, the concentration of cadmium in the hemp oil is less than about 5 μg/kg, less than about 6 μg/kg, less than about 7 μg/kg, less than about 8 μg/kg, less than about 9 μg/kg, or less than about 10 μg/kg. In some embodiments, the concentration of cadmium in the hemp oil is less than about 7 μg/kg.

In certain embodiments, the concentration of mercury in the hemp oil is less than about 1 μg/kg, less than about 2 μg/kg, less than about 3 μg/kg, less than about 4 μg/kg, less than about 5 μg/kg, or less than about 6 μg/kg. In some embodiments, the concentration of arsenic in the hemp oil is less than about 2 μg/kg.

In certain embodiments, the concentration of lead in the hemp oil is less than about 25 μg/kg, less than about 26 μg/kg, less than about 27 μg/kg, less than about 28 μg/kg, less than about 29 μg/kg, or less than about 30 μg/kg. In some embodiments, the concentration of lead in the hemp oil is less than about 27 μg/kg.

In certain embodiments, the hemp oil comprises less than about 2 parts per billion of aflatoxin. In certain embodiments, the hemp oil comprises less than about 3 parts per billion of ochratoxin. In certain embodiments, the hemp oil comprises less than about 0.2 parts per billion of abamectin B1a. In certain embodiments, the hemp oil comprises less than about 0.2 parts per billion of abamectin B1b. In certain embodiments, the hemp oil comprises less than about 0.1 parts per billion of azoxystrobin. In certain embodiments, the hemp oil comprises less than about 0.1 parts per billion of bifenazate. In certain embodiments, the hemp oil comprises less than about 0.2 parts per billion of bifenthrin. In certain embodiments, the hemp oil comprises less than about 0.5 parts per billion of cyfluthrin. In certain embodiments, the hemp oil comprises less than about 10 parts per billion of daminozide. In certain embodiments, the hemp oil comprises less than about 0.1 parts per billion of etoxazole. In certain embodiments, the hemp oil comprises less than about 0.1 parts per billion of fenoxycarb. In certain embodiments, the hemp oil comprises less than about 0.1 parts per billion of imazalil. In certain embodiments, the hemp oil comprises less than about 0.1 parts per billion of imidacloprid. In certain embodiments, the hemp oil comprises less than about 0.1 parts per billion of myclobutanil. In certain embodiments, the hemp oil comprises less than about 0.1 parts per billion of paclobutrazol. In certain embodiments, the hemp oil comprises less than about 0.1 parts per billion of piperonyl butoxide. In certain embodiments, the hemp oil comprises less than about 0.1 parts per billion of pyrethrin. In certain embodiments, the hemp oil comprises less than about 0.1 parts per billion of spinosad. In certain embodiments, the hemp oil comprises less than about 0.1 parts per billion of spiromesifen. In certain embodiments, the hemp oil comprises less than about 0.1 parts per billion of spirotetramat. In certain embodiments, the hemp oil comprises less than about 0.1 parts per billion of trifloxystrobin.

In certain embodiments, the hemp oil comprises less than about 3300 parts per million of isopropanol.

In certain embodiments, the compounds provided herein can be administered as the sole therapeutically active agent, or they can be administered in combination with other therapeutically active agents.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

In one aspect, the topical pharmaceutical compositions described herein may be used for the treatment of neuropathic and nociceptive pain.

In another aspect, the topical pharmaceutical compositions described herein may be used for the treatment of peripheral neuropathy.

In various embodiments, the method comprising administering to the patient an effective amount of a topical pharmaceutical composition, wherein the topical pharmaceutical composition comprises:

(i) lidocaine or a pharmaceutically acceptable salt thereof,

(ii) capsaicin;

(iii) hemp oil; and

(iv) a pharmaceutically acceptable carrier.

In various embodiments, the method comprising administering to the patient an effective amount of a topical pharmaceutical composition, wherein the topical pharmaceutical composition comprises:

(i) about 3% (w/w) to about 5% (w/w) lidocaine or a pharmaceutically acceptable salt thereof;

(ii) about 1% (w/w) to about 2% (w/w) capsaicin;

(iii) about 0.2% (w/w) to about 3% (w/w) hemp oil; and

(iv) a pharmaceutically acceptable carrier,

wherein the weight percentages are based on the total weight of the topical pharmaceutical composition.

In various embodiments, the method comprising administering to the patient an effective amount of a topical pharmaceutical composition, wherein the topical pharmaceutical composition comprises:

(i) about 3% (w/w) to about 5% (w/w) lidocaine hydrochloride;

(ii) about 1% (w/w) to about 2% (w/w) capsaicin;

(iii) about 0.2% (w/w) to about 3% (w/w) hemp oil;

(iv) about 1% (w/w) to about 3% (w/w) N-palmitoylethanolamine;

(v) about 3% (w/w) to about 5% (w/w) Acmella oleracea extract;

(vi) about 8% (w/w) to about 12% (w/w) methylsulfonylmethane;

(vii) about 1% (w/w) to about 3% (w/w) geranium oil;

(viii) about 1% (w/w) to about 3% (w/w) lavender oil; and

(ix) about 60% (w/w) to about 85% (w/w) of an emulsified lotion base,

wherein the weight percentages are based on the total weight of the topical pharmaceutical composition.

In various embodiments, the method comprising administering to the patient an effective amount of a topical pharmaceutical composition, wherein the topical pharmaceutical composition comprises:

(i) about 4% (w/w) lidocaine hydrochloride;

(ii) about 1.5% (w/w) capsaicin;

(iii) about 2.5% (w/w) hemp oil;

(iv) about 2% (w/w) N-palmitoylethanolamine;

(v) about 4% (w/w) Acmella oleracea extract;

(vi) about 10% (w/w) methylsulfonylmethane;

(vii) about 2% (w/w) geranium oil;

(viii) about 2% (w/w) lavender oil; and

(ix) about 72% (w/w) of an emulsified lotion base,

wherein the weight percentages are based on the total weight of the topical pharmaceutical composition.

In various embodiments, the method comprising administering to the patient an effective amount of a topical pharmaceutical composition, wherein the topical pharmaceutical composition comprises:

(i) about 4% (w/w) lidocaine hydrochloride;

(ii) about 1.5% (w/w) capsaicin;

(iii) about 0.4% (w/w) hemp oil;

(iv) about 2% (w/w) N-palmitoylethanolamine;

(v) about 4% (w/w) Acmella oleracea extract;

(vi) about 10% (w/w) methylsulfonylmethane;

(vii) about 2% (w/w) geranium oil;

(viii) about 2% (w/w) lavender oil; and

(ix) about 74.1% (w/w) of an emulsified lotion base,

wherein the weight percentages are based on the total weight of the topical pharmaceutical composition.

The topical pharmaceutical compositions provided herein may also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

In certain embodiments, the method further comprises administering to the patient a second therapeutically active agent. In some embodiments, the topical pharmaceutical compositions described herein and the second therapeutically active agent are administered simultaneously, together or separately, or separately at different times, as part of a regimen.

When administered for the treatment or prevention of a disease or disorder disclosed herein, it may be understood that an effective dosage can vary depending upon many factors such as the particular compound or therapeutic combination utilized, the mode of administration, and severity of the condition being treated, as well as the various physical factors related to the individual being treated. In therapeutic applications, a pharmaceutical composition described herein may be provided to a patient already suffering from said disease or disorder in an amount sufficient to cure or at least partially ameliorate the symptoms of the disease or disorder and its complications. The dosage to be used in the treatment of a specific individual typically must be subjectively determined by an attending physician. The variables involved include the specific condition and state as well as the size, age and response pattern of the patient. In some embodiments, the amount sufficient to cure or at least partially ameliorate the symptoms of the disease or disorder and its complications is an effective amount. In some embodiments, the amount sufficient to cure or at least partially ameliorate the symptoms of the disease or disorder and its complications is a therapeutically effective amount.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

### Example 1: Manufacturing Process for Topical Pharmaceutical Compositions Comprising 75 mg and 450 mg Cannabidiol

In the following example, the manufacturing process is outlined for all exemplified dosage strengths.

The corresponding amounts of the ingredients are provided in the formulas under Examples 1.1 and 1.2 below.

### Manufacture of the Topical Pharmaceutical Composition

The emulsified lotion base was partially melted and then separated into two equal portions (by weight).

The hemp oil is then added to the first portion of the partially melted emulsified lotion base to create a mixture. The mixture was then heated until the emulsified lotion base had completely melted. Thereafter the second portion of the partially melted emulsified lotion base was added to the mixture.

The lidocaine hydrochloride, capsaicin, methylsulfonylmethane, Acmella oleracea extract, geranium oil, N-palmitoylethanolamine, and lavender oil were then added to the mixture and stirred until a homogenous mixture was obtained.

The final topical pharmaceutical composition was then produced by cooling the mixture, with continual stirring.

### Example 1.1

TABLE 1
Formula of Topical Pharmaceutical Composition
Comprising 450 mg Cannabidiol

| Ingredients | wt %* |
|---|---|
| Lidocaine hydrochloride | 4 |
| Capsaicin^{a} | 1.5 |
| Hemp oil^{b} | 2.5 |
| Methylsulfonylmethane | 10 |
| Acmella Oleracea extract^{c} | 4 |
| Geranium oil^{d} | 2 |
| N-palmitoylethanolamine | 2 |
| Lavender oil^{e} | 2 |
| Emulsified lotion base (aloe vera juice, shea butter, | 72 |
| emulsifying wax, vegetable glycerin, stearic acid, | |
| silver dihydrogen citrate, citric acid) | |
| Total | 100 |

*based upon the total weight of the topical pharmaceutical formulation
^{a}capsaicin purchased from hotsauce.com
^{b}hemp oil purchased from Colorado Wholesale Company
^{c}Acmella Oleracea extract purchased from Hawaii Pharm
^{d}geranium oil purchased from Zongle Therapeutics
^{e}lavender oil purchased from Zongle Therapeutics

### Example 1.2

TABLE 1
Formula of Topical Pharmaceutical Composition
Comprising 75 mg Cannabidiol

| Ingredients | wt %* |
|---|---|
| Lidocaine hydrochloride | 4 |
| Capsaicin^{a} | 1.5 |
| Hemp oil^{b} | 0.4 |
| Methylsulfonylmethane | 10 |
| Acmella Oleracea extract^{c} | 4 |
| Geranium oil^{d} | 2 |
| N-palmitoylethanolamine | 2 |
| Lavender oil^{e} | 2 |
| Emulsified lotion base (aloe vera juice, shea butter, | 74.1 |
| emulsifying wax, vegetable glycerin, stearic acid, | |
| silver dihydrogen citrate, citric acid) | |
| Total | 100 |

^{a}capsaicin purchased from hotsauce.com
^{b}hemp oil purchased from Colorado Wholesale Company
^{c}Acmella Oleracea extract purchased from Hawaii Pharm
^{d}geranium oil purchased from Zongle Therapeutics
^{e}lavender oil purchased from Zongle Therapeutics

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A topical pharmaceutical composition comprising:
(i) lidocaine or a pharmaceutically acceptable salt thereof;
(ii) capsaicin;
(iii) hemp oil; and
(iv) a pharmaceutically acceptable carrier.

2. The topical pharmaceutical composition of claim 1, wherein the topical pharmaceutical composition comprises from about 3% (w/w) to about 5% (w/w) lidocaine or a pharmaceutically acceptable salt thereof, wherein the weight percentages are based on the total weight of the topical pharmaceutical composition.

3. The topical pharmaceutical composition of claim 1, wherein the topical pharmaceutical composition comprises from about 1% (w/w) to about 2% (w/w) capsaicin,
wherein the weight percentages are based on the total weight of the topical pharmaceutical composition.

4. The topical pharmaceutical composition of claim 1, wherein the topical pharmaceutical composition comprises from about 0.2% (w/w) to about 3% (w/w) hemp oil,
wherein the weight percentages are based on the total weight of the topical pharmaceutical composition.

5. A topical pharmaceutical composition comprising:
(i) about 3% (w/w) to about 5% (w/w) lidocaine or a pharmaceutically acceptable salt thereof;
(ii) about 1% (w/w) to about 2% (w/w) capsaicin;
(iii) about 0.2% (w/w) to about 3% (w/w) hemp oil; and
(iv) a pharmaceutically acceptable carrier,
wherein the weight percentages are based on the total weight of the topical pharmaceutical composition.

6. The topical pharmaceutical composition of claim 1, wherein the topical pharmaceutical composition further comprises N-palmitoylethanolamine.

7. The topical pharmaceutical composition of claim 6, wherein the topical pharmaceutical composition comprises from about 1% (w/w) to about 3% (w/w) N-palmitoylethanolamine, wherein the weight percentages are based on the total weight of the topical pharmaceutical composition.

8. The topical pharmaceutical composition of claim 1, wherein the topical pharmaceutical composition further comprises Acmella oleracea extract.

9. The topical pharmaceutical composition of claim 8, wherein the topical pharmaceutical composition comprises from about 3% (w/w) to about 5% (w/w) Acmella oleracea extract, wherein the weight percentages are based on the total weight of the topical pharmaceutical composition.

10. The topical pharmaceutical composition of claim 1, wherein the topical pharmaceutical composition further comprises methylsulfonylmethane.

11. The topical pharmaceutical composition of claim 10, wherein the topical pharmaceutical composition comprises from about 8% (w/w) to about 12% (w/w) methylsulfonylmethane, wherein the weight percentages are based on the total weight of the topical pharmaceutical composition.

12. The topical pharmaceutical composition of claim 1, wherein the topical pharmaceutical composition further comprises geranium oil.

13. The topical pharmaceutical composition of claim 12, wherein the topical pharmaceutical composition comprises from about 1% (w/w) to about 3% (w/w) geranium oil,
wherein the weight percentages are based on the total weight of the topical pharmaceutical composition.

14. The topical pharmaceutical composition of claim 1, wherein the topical pharmaceutical composition further comprises lavender oil.

15. The topical pharmaceutical composition of claim 14, wherein the topical pharmaceutical composition comprises from about 1% (w/w) to about 3% (w/w) lavender oil, wherein the weight percentages are based on the total weight of the topical pharmaceutical composition.

16. The topical pharmaceutical composition of claim 1, wherein the topical pharmaceutical composition comprises from about 60% (w/w) to about 85% (w/w) of the pharmaceutically acceptable carrier, wherein the weight percentages are based on the total weight of the topical pharmaceutical composition.

17. The topical pharmaceutical composition of claim 1, wherein the pharmaceutically acceptable carrier is an emulsified lotion base.

18. The topical pharmaceutical composition of claim 1, wherein the pharmaceutically acceptable salt of lidocaine is a hydrochloride salt.

19. A topical pharmaceutical composition comprising:
(i) about 3% (w/w) to about 5% (w/w) lidocaine hydrochloride;
(ii) about 1% (w/w) to about 2% (w/w) capsaicin;
(iii) about 0.2% (w/w) to about 3% (w/w) hemp oil;
(iv) about 1% (w/w) to about 3% (w/w) N-palmitoylethanolamine;
(v) about 3% (w/w) to about 5% (w/w) Acmella oleracea extract;
(vi) about 8% (w/w) to about 12% (w/w) methylsulfonylmethane;
(vii) about 1% (w/w) to about 3% (w/w) geranium oil;
(viii) about 1% (w/w) to about 3% (w/w) lavender oil; and
(ix) about 60% (w/w) to about 85% (w/w) of an emulsified lotion base,
wherein the weight percentages are based on the total weight of the topical pharmaceutical composition.

20. A topical pharmaceutical composition comprising:
(i) about 4% (w/w) lidocaine hydrochloride;
(ii) about 1.5% (w/w) capsaicin;
(iii) about 2.5% (w/w) hemp oil;
(iv) about 2% (w/w) N-palmitoylethanolamine;
(v) about 4% (w/w) Acmella oleracea extract;
(vi) about 10% (w/w) methylsulfonylmethane;
(vii) about 2% (w/w) geranium oil;
(viii) about 2% (w/w) lavender oil; and
(ix) about 72% (w/w) of an emulsified lotion base,
wherein the weight percentages are based on the total weight of the topical pharmaceutical composition.

21. A topical pharmaceutical composition comprising:
(i) about 4% (w/w) lidocaine hydrochloride;
(ii) about 1.5% (w/w) capsaicin;
(iii) about 0.4% (w/w) hemp oil;
(iv) about 2% (w/w) N-palmitoylethanolamine;
(v) about 4% (w/w) Acmella oleracea extract;
(vi) about 10% (w/w) methylsulfonylmethane;
(vii) about 2% (w/w) geranium oil;
(viii) about 2% (w/w) lavender oil; and
(ix) about 74.1% (w/w) of an emulsified lotion base, wherein the weight percentages are based on the total weight of the topical pharmaceutical composition.

22. The topical pharmaceutical composition of claim 1, wherein the hemp oil comprises one or more cannabinoids; or
the topical pharmaceutical composition comprises from about 0.69% (w/w) to about 0.79% (w/w) cannabidiol; or
the topical pharmaceutical composition comprises from about 0.02% (w/w) to about 0.04% (w/w) cannabichromene; or
the topical pharmaceutical composition comprises less than about 0.3% (w/w) Δ⁹-tetrahydrocannabinol,
wherein the weight percentage is based on the total weight of the topical pharmaceutical composition.

23.-25. (canceled)

26. A method of treating neuropathic and nociceptive pain in a patient in need thereof comprising administering to the patient an effective amount of a topical pharmaceutical composition of claim 1.

27. (canceled)
